(19) United States
(12) Patent Application Publication
(10) Pub. No.: US 2022/0313339 A1
(43) Pub. Date: Oct. 6, 2022

(54) SPINAL PERCUTANOUS PUNCTURE PLASMA SCALPEL HEAD AND OPERATION METHOD OF SPINAL PERCUTANEOUS PUNCTURE PLASMA SCALPEL HEAD

(71) Applicant: Beijing Longfu Hospital, Beijing (CN)

(72) Inventors: Yanli LU, Beijing (CN); Yuanli WANG, Beijing (CN); Qingpu WANG, Beijing (CN); Zhijun TIAN, Beijing (CN)

(21) Appl. No.: 17/596,761

(22) PCT Filed: Jun. 12, 2020
(86) PCT No.: PCT/CN2020/095876

§ 371 Date: Dec. 17, 2021

(30) Foreign Application Priority Data
Jun. 19, 2019 (CN) 201910533548.5

(51) Int. Cl. A61B 18/04

(52) U.S. Cl. CPC A61B 18/04; A61B 2018/00339

(57) ABSTRACT

A plasma scalpel head for spinal percutaneous puncture and an operation method of the plasma scalpel head for spinal percutaneous puncture are provided. The plasma scalpel head for spinal percutaneous puncture includes a needle core. The needle core includes a main ablation electrode and a needle core body. The main ablation electrode is arranged at an ablation end of the plasma scalpel head. The needle core body is externally provided with a first insulating layer. The first insulating layer is externally provided with a reflow electrode layer. A part of the first insulating layer that is not covered with the reflow electrode layer forms a main ablation electrode protection insulating ring. The reflow electrode layer is provided thereon with a second insulating layer, and a part of the reflow electrode layer that is not covered with the second insulating layer forms a reflow electrode.

### CROSS-REFERENCE TO RELATED APPLICATION

The present invention is a national stage application of International Patent Application No. PCT/CN2020/095876, which is filed on Jun. 12, 2020, and claims the priority of Chinese patent application No. 201910533548.5, which is filed on Jun. 19, 2019, entitled “Plasma scalpel head for spinal percutaneous puncture and Operation Method of Plasma scalpel head for spinal percutaneous puncture”, which is incorporated in its entirety herein by reference.

### TECHNICAL FIELD

The disclosure relates to a technical field of plasma scalpels, in particular to a plasma scalpel head for spinal percutaneous puncture and an operation method of the plasma scalpel head for spinal percutaneous puncture.

### BACKGROUND

A low-temperature plasma surgical system excites a conductive liquid at a tip of a scalpel head into a plasma state through an electric field and the scalpel head, and applies a corresponding voltage to the plasma according to different working conditions, so that charged particles therein have a certain kinetic energy to break a target tissue and make it cracked at a molecular level so as to generate effects of vaporization, cutting, ablation, and hemostasis. Since the electric field does not directly act on the tissue, so excess heat may be avoided, thereby reducing thermal damage to surrounding normal tissues to a greatest extent. The plasma scalpel head at this stage still has a defect that it cannot continuously generate a large amount of plasma, which increases the difficulty of ablating nucleus pulposus, which will bring a great uncertainty to a surgery, increase the risk of the surgery, and easily produce complications, and so on.

Considering the structure of the existing plasma scalpel heads, the plasma scalpel heads in the prior art are mostly made of platinum, and most of the plasma scalpel heads have complicated structures and high costs; further, many plasma scalpel heads have restriction of single use, so that the plasma scalpel heads are expensive in price and limited in the number of times of use.

Considering the operation method of the existing plasma scalpel head, since the existing plasma scalpel head needs to enter in different directions many times during a treatment, so that the operation time is relatively long and the patient suffers more pain, and the working efficiency of the spinal percutaneous puncture plasma surgery is low.

### SUMMARY

In view of the above analysis, embodiments of the disclosure aim to provide a plasma scalpel head for spinal percutaneous puncture and an operation method of the plasma scalpel head for spinal percutaneous puncture, so as to solve the technical problems that the existing plasma scalpel head for spinal percutaneous puncture and the plasma scalpel employ expensive platinum material scalpel heads, and the scalpel is complicated in structure and high in manufacturing cost, and the like.

The purpose of the disclosure is mainly achieved through the following technical solutions.

On the one hand, the disclosure discloses a plasma scalpel head for spinal percutaneous puncture, the plasma scalpel head includes a needle core. The needle core includes a main ablation electrode and a needle core body. The main ablation electrode is arranged at an ablation end of the plasma scalpel head. The needle core body is externally provided with a first insulating layer with a same length as the needle core body. The first insulating layer is externally provided with a reflow electrode layer. A length of the reflow electrode layer is less than a length of the first insulating layer, and a part of the first insulating layer that is not covered with the reflow electrode layer forms a main ablation electrode protection insulating ring.

The reflow electrode layer is provided thereon with a second insulating layer having a length less than the length of the reflow electrode layer, and a part of the reflow electrode layer that is not covered with the second insulating layer forms a reflow electrode.

In some embodiments, a length of the main ablation electrode protection insulating ring is in a range of 1 mm to 1.5 mm. An angle between the main ablation electrode and the needle core body is in a range of 10 degrees to 15 degrees.

In some embodiments, a material of the needle core of the plasma scalpel head is stainless steel; and the ablation end of the main ablation electrode is of cylindrical-shaped.

In some embodiments, a material of the needle core of the plasma scalpel head is tungsten steel; and the ablation end of the main ablation electrode is of cone-shaped.

In some embodiments, a ratio of a surface area of the main ablation electrode of the plasma scalpel to a surface area of the reflow electrode of the plasma scalpel is in a range of 1:3 to 1:7.

In some embodiments, both the first insulating layer and the second insulating layer are made of plastic insulating layers.

On the other hand, a plasma scalpel kit for spinal percutaneous puncture is provided, the plasma scalpel kit is used in cooperation with the plasma scalpel head above-mentioned, and the plasma scalpel kit includes a puncture needle core and a puncture needle sleeved on the puncture needle core, the puncture needle is used for puncturing and being placed into a diseased site of a spine.

In yet another aspect, the disclosure further discloses an operation method of a plasma scalpel head for spinal percutaneous puncture, which employs the above-mentioned plasma scalpel head and plasma scalpel kit, and the operation method of the plasma scalpel head includes the following steps.
step S1, the puncture needle is used to be placed in the diseased disc and then the plasma scalpel head enters the nucleus pulposus from the puncture needle.
step S2, the plasma scalpel head and the puncture needle are locked, and the needle core of the plasma scalpel head is rotated by 360 degrees to form a first cone ablation region; and the first cone ablation region and peripheral nucleus pulposus tissue are vaporized and ablated by the main ablation electrode so as to achieve intervertebral disc decompression and intervertebral disc formation.

In some embodiments, the operation method of the above mentioned plasma scalpel head for spinal percutaneous puncture further includes step S3, in which the puncture needle and the plasma scalpel head are retracted by 1 mm to 2 mm, and the needle core of the plasma scalpel head is rotated by 360 degrees to form a second cone ablation region; and the second cone ablation region and peripheral nucleus pulposus tissue are vaporized and ablated by the main ablation electrode so as to achieve intervertebral disc decompression and intervertebral disc formation.

In some embodiments, the operation method of the above mentioned plasma scalpel head for spinal percutaneous puncture further includes step S4, in which the puncture needle and the plasma scalpel head are retracted again by 1 mm to 2 mm, and the needle core of the plasma scalpel head is rotated by 360 degrees to form a third cone ablation region, and the third ablation region and peripheral nucleus pulposus tissue are ablated and vaporized by the main ablation electrode so as to achieve intervertebral disc decompression and intervertebral disc formation.

Compared with the prior art, the disclosure may achieve at least one of the following advantageous effects.
(1) In the disclosure, the first insulating layer is directly arranged outside the needle core, and the reflow electrode layer is arranged outside the first insulating layer. The first insulating layer and the reflow electrode layer form the main ablation electrode protection insulating ring near the ablation end, and the main ablation electrode protection insulating ring separates the main ablation electrode from the reflow electrode to form a certain protection distance so as to ensure that the main ablation electrode emits plasma normally; the existing plasma scalpel head is provided with the first insulating layer, the second insulating layer, and the third insulating layer outside the needle core, and an insulating ring and a heating part need to be separately provided between the main ablation electrode and the reflow electrode, so that the structure is quite complicated and expensive in cost, which is not conducive to mass production.
(2) This disclosure controls the angle between the main ablation electrode and the needle core body to be between 10 degrees and 15 degrees. After the plasma scalpel enters the cervical nucleus pulposus, the plasma scalpel head is rotated by 360 degrees, and the rotational trajectory of the plasma scalpel head can form a cone plasma ablation region. This design can not only increase the ablation range of the cervical nucleus pulposus and reduce the internal pressure of the cervical nucleus pulposus, but also can avoid multiple entries of the plasma scalpel head in different directions, reducing surgery time, alleviating the pain of patients and at the same time enabling to ensure efficient work of the spinal percutaneous puncture plasma surgery.
(3) From an economical point of view, the needle cores of the existing plasma scalpels are mostly made of platinum, which makes the plasma scalpel head expensive; while the use of stainless steel or tungsten steel in this disclosure can greatly reduce production cost of the plasma scalpel heads and the surgery cost, and is beneficial to encouraging more patients to accept the treatment of the plasma surgery.

In this disclosure, the above-mentioned technical solutions may further be combined with each other to achieve more preferred combination solutions. Other features and advantages of the disclosure will be described in the following specification, and some of the advantages may become apparent from the specification, or be understood by implementing the disclosure. The purpose and other advantages of the disclosure may be realized and obtained through the embodiments of the specification and the contents specified in the accompanying drawings.

### BRIEF DESCRIPTION OF THE DRAWINGS

The drawings are only for the purpose of illustrating specific embodiments, and are not to be considered as a limitation to the disclosure. Throughout the drawings, the same reference numerals refer to the same components.

FIG. 1 is a front view of a plasma scalpel head for spinal percutaneous puncture according to embodiment 1 of the disclosure.

FIG. 2 is a schematic structural diagram of a plasma scalpel head for spinal percutaneous puncture according to embodiment 1 of the disclosure.

### REFERENCE NUMERALS

1: Main ablation electrode; 2: Main ablation electrode protection insulating ring; 3: Reflow electrode; 4: First insulating layer; 5: Reflow electrode layer; 6: Second insulating layer.

### DETAILED DESCRIPTION OF THE EMBODIMENTS

The preferred embodiments of the disclosure are described in detail below with reference to the accompanying drawings. The drawings form a part of the disclosure and serve to explain the principle of the disclosure with the embodiments of the disclosure, and are not intended to limit the scope of the disclosure.

### Embodiment 1

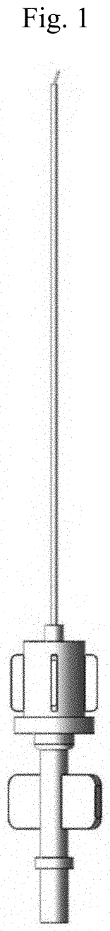

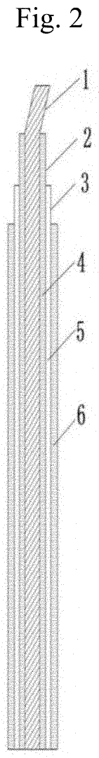

The embodiment discloses a plasma scalpel head for spinal percutaneous puncture, as shown in FIG. 1 and FIG. 2, the plasma scalpel head includes a needle core; the needle core includes a main ablation electrode 1 and a needle core body; the main ablation electrode 1 is arranged at an ablation end of the plasma scalpel head; the needle core body is externally provided with a first insulating layer 4 with the same length as the needle core body; the first insulating layer 4 is externally provided with a reflow electrode layer 5; the length of the reflow electrode layer 5 is less than the length of the first insulating layer 4, and a part of the first insulating layer 4 that is not covered with the reflow electrode layer 5 is a main ablation electrode protection insulating ring 2; the reflow electrode layer 5 is provided thereon with a second insulating layer 6 with a length less than a length of the reflow electrode layer 5, and a part of the reflow electrode layer 5 that is not covered with the second insulating layer 6 is the reflow electrode 3.

Specifically, the plasma scalpel head includes the needle core and a three-layer structure arranged outside the needle core, the needle core includes the main ablation electrode 1 and the needle core body which are integrally together, the main ablation electrode 1 is the ablation end of the plasma scalpel head; the three-layer structure includes the first insulating layer 4, the reflow electrode layer 5, and the second insulating layer 6, respectively; the needle core body is provided thereon with the first insulating layer 4 with the same length as the needle core body, the first insulating layer 4 is provided thereon with the reflow electrode layer 5 whose length is less than the length of the first insulating layer 4. The part of the first insulating layer 4 that is not covered with the reflow electrode layer 5 is the main ablation electrode protection insulating ring 2 which is configured to form a certain insulation protection distance between the main ablation electrode 1 and the reflow electrode layer 5, so that the main ablation electrode 1 can generate plasma. When the plasma scalpel head enters the nucleus pulposus, the plasma can vaporize the nucleus pulposus tissue at a low temperature, thereby ablating the nucleus pulposus tissue to achieve intervertebral disc decompression and intervertebral disc formation.

Compared with the prior art, in the disclosure, the needle core is externally and directly provided with the first insulating layer 4, and the first insulating layer 4 is externally provided with the reflow electrode layer 5, the first insulating layer 4 and the reflow electrode layer 5 form the main ablation electrode protection insulating ring 2 near the ablation end, and the main ablation electrode protection insulating ring 2 separates the main ablation electrode 1 from the reflow electrode layer 5 to form a certain protection distance so as to ensure that the main ablation electrode 1 emits plasma normally; the insulation structure of the plasma scalpel head in prior art is quite complicated, the existing plasma scalpel head is provided with the first insulating layer 4, the second insulating layer 6, and the third insulating layer outside the needle core, and the insulating ring and the heating part need to be separately provided between the main ablation electrode 1 and the reflow electrode layer 5, so that the plasma scalpel head is quite complicated in structure and expensive in cost, which is not conducive to mass production.

In order to form a certain insulation protection distance between the main ablation electrode 1 and the reflow electrode layer 5, the length of the main ablation electrode protection insulating ring 2 is 1 mm to 1.5 mm; the length of the main ablation electrode protection insulating ring 2 is controlled to be 1 mm to 1.5 mm, so that the main ablation electrode 1 is prevented from being in contact with the reflow electrode layer 5, and it may be ensured that the main ablation electrode 1 generates plasma normally. The plasma breaks molecular bonds of the tissue at a lower temperature, causing molecules to be lysed, and then vaporizes cervical nucleus pulposus or lumbar nucleus pulposus for the cervical disc decompression or the intervertebral disc decompression.

In order to increase the range of ablation of the nucleus pulposus and reduce the pressure in the nucleus pulposus, the angle between the main ablation electrode 1 and the needle core body is controlled between 10 degrees and 15 degrees. Specifically, the angle between the main ablation electrode 1 and the needle core body are set to be 10 degrees to 15 degrees, after the plasma scalpel enters the cervical nucleus pulposus, the plasma scalpel head is rotated by 360 degrees, and the rotational trajectory of the plasma scalpel head can form a cone plasma ablation region. Controlling the angle between the main ablation electrode 1 and the needle core body to be between 10 degrees and 15 degrees can not only increase the ablation range of the cervical nucleus pulposus and reduce the pressure in the cervical nucleus pulposus, but also can avoid multiple entries of the plasma scalpel head in different directions, reducing surgery time, alleviating the pain of patients and at the same time enabling to ensure the efficient work of the spinal percutaneous plasma surgery.

In order to reduce production costs and ensure that the plasma scalpel head can generate a large amount of plasma, the needle core of the plasma scalpel head is made of stainless steel; the needle core being designed to be stainless steel is because, on the one hand, the stainless steel is resistant to ablation, ionization and high temperature, meeting the requirement of being able to excite a large amount of plasma, on the other hand, the needle cores of the existing plasma scalpels are mostly made of platinum, which makes the plasma scalpel head expensive, while the needle core made of stainless steel can greatly reduce the production cost of the plasma scalpel head, thereby reducing plasma surgery costs, which may encourage more patients to accept the treatment of the plasma surgery from an economical point of view.

It is to be noted that in order to ensure that the plasma scalpel head can continuously generate a large amount of plasma, the ablation end of the main ablation electrode 1 has a cylindrical shape. Specifically, the main ablation electrode 1 being arranged in a shape of a cylinder is because: with the tip effect, the curvature of the tip is big, the charge density is high, the nearby field strength is strong, and therefore the field intensity near the main ablation electrode 1 is larger than the electric field of the existing plasma scalpel head so that a large amount of plasma can be excited. It is to be noted that the plasma emitted by the cylindrical main ablation electrode 1 can form a cluster-like plasma cluster, which can break the molecular bonds of a target tissue with the most appropriate kinetic energy, so that the target tissue is lysed at a molecular level, and can be accurately vaporized, cut, ablated and hemostatic. Since the electric field generated by the plasma does not directly act on the tissue, excessive heat can be avoided so as to minimize the thermal damage to surrounding normal tissues to a greatest extent.

Similarly, in order to reduce the production cost and ensure that the plasma scalpel head can generate a large amount of plasma, the needle core of the plasma scalpel head is made of tungsten steel. The needle core being designed to be made of tungsten steel is because, on the one hand, the tungsten steel is resistant to ablation, ionization and high temperature, and the melting point of tungsten steel reaches 6000 degrees Celsius, meeting the requirement of being able to excite a large amount of plasma, on the other hand, the needle cores of the existing plasma scalpels are mostly made of platinum, which makes the plasma scalpel head expensive, while the needle cores made of tungsten steel can greatly reduce the production cost of the plasma scalpel heads, thereby reducing plasma surgery costs, which may encourage more patients to accept the treatment of the plasma surgery from an economical point of view.

It is to be noted that when the needle core is made of tungsten steel, the ablation end of the main ablation electrode 1 has a cone shape, and the use of a cone-shaped needle core is not only easy to process, but also low in cost.

In order to control the plasma emitted by the main ablation electrode 1 to be within a certain distance, the ratio of the surface area of the main ablation electrode 1 of the plasma scalpel head to the surface area of the reflow electrode layer 5 of the plasma scalpel head is in a range of 1:3 to 1:7. Specifically, the larger the surface areas of the main ablation electrode 1 and the reflow electrode layer 5, the smaller the voltage intensity through their surfaces, the easier it is to form a voltage potential flow. In order to control the plasma emission range of the main ablation electrode 1, the ratio of the surface area of the main ablation electrode 1 to the surface area of the reflow electrode layer 5 is in the range of 1:3-1:7. On the one hand, the emission range of the plasma and the ablation effect of the main ablation electrode 1 can be ensured, on the other hand, from a safety perspective, if the ratio of the surface area of the main ablation electrode 1 to the surface area of the reflow electrode layer 5 exceeds 1:3, the pressure intensity of the main ablation electrode 1 is likely to be too big, which is easy to adversely affect the main ablation electrode 1, and at the same time also has an adverse effect on the operated object.

In order to achieve multiple times of use of the plasma scalpel head and ensure the safety of operators, both the first insulating layer 4 and the second insulating layer 6 are made of plastic insulating layers.

Specifically, the first insulating layer 4 and the second insulating layer 6 in the embodiment are made of insulating plastics. The first insulating layer 4 employs a plastic insulating layer instead of existing silica gel insulating material that is easily ablated in the field strength formed by the main ablation electrode 1 and the reflow electrode layer 5, while the use of the plastic insulating layer can effectively solve the problem of ablation of the insulating ring, providing an effective guarantee for multiple times of use of the plasma scalpel head. Further, considering operational safety of healthcare professionals and usage cost, materials of the first insulating layer 4 and the second insulating layer being designed as plastic insulation can not only save costs, but also ensure the surgery safety.

In order to make the needle core be as thick as possible, in this embodiment, the first insulating layer 4 is thickened and expanded, so as to exchange volume for effective insulation duration.

### Embodiment 2

The embodiment provides an operation method of a plasma scalpel head for spinal percutaneous puncture, using the plasma scalpel head provided in Embodiment 1 and the plasma scalpel kit, and the operation method of the plasma scalpel head for spinal percutaneous puncture used in cervical vertebra includes the following steps.
Step S1, the puncture needle is employed to be placed into the diseased cervical disc and then the plasma scalpel head enters the nucleus pulposus through the puncture needle.
Step S2, the plasma scalpel head and the puncture needle are locked, and the needle core of the plasma scalpel head is rotated by 360 degrees to form a first cone ablation region; and the first cone ablation region and peripheral nucleus pulposus tissue are vaporized and ablated at a low temperature.
Step S3, the puncture needle and the plasma scalpel head are retracted by 1 mm to 2 mm, and the needle core of the plasma scalpel head is rotated by 360 degrees to form a second cone ablation region; and the second cone ablation region and peripheral nucleus pulposus tissue are vaporized and ablated at a low temperature.
Step S4, the puncture needle and the plasma scalpel head are retracted again by 1 mm to 2 mm, and the needle core of the plasma scalpel head is rotated by 360 degrees to form a third cone ablation region; an ablation foot pedal is pressed to ablate the third cone ablation region and peripheral nucleus pulposus tissue.

Specifically, before the step S1, it is necessary to be in a supine posture, the neck is slightly extended, a Kirschner wire locates in vitro the diseased space under fluoroscopy, the diseased space is marked and then local anesthesia is performed, the anterolateral approach is, guided by a C arm, between an arterial sheath and a visceral sheath, in the step S1, the puncture needle is placed in the center of the intervertebral disc, which is at the midpoint in anterior-posterior and lateral planes in the fluoroscopy; the puncture needle core is pulled out, the plasma scalpel head is inserted and pushed forward, it is to be noted that the tip of the plasma scalpel head does not extend out of the puncture needle, the plasma scalpel head is kept still and the puncture needle is pulled back, the main ablation electrode 1 of the plasma scalpel head is monitored exposed under fluoroscopy, the plasma scalpel head and the puncture needle are locked, and the position of the plasma scalpel head is confirmed again; in the step S2, the needle core of the plasma scalpel head is rotated by 360 degrees to form a first cone ablation region; the first cone ablation region and peripheral nucleus pulposus tissue are vaporized and ablated at a low temperature; in the step S3, the puncture needle and the plasma scalpel head are retracted by 1 mm to 2 mm, and the needle core of the plasma scalpel head is rotated by 360 degrees to form a second cone ablation region; and the second cone ablation region and peripheral nucleus pulposus tissue are vaporized and ablated at a low temperature; in the step S4, the puncture needle and the plasma scalpel head are retracted again by 1 mm to 2 mm, and the needle core of the plasma scalpel head is rotated by 360 degrees to form a third cone ablation region; the ablation foot pedal is pressed to ablate the third cone ablation region and peripheral nucleus pulposus tissue; after the ablation, the puncture needle and plasma scalpel head are pulled out to complete the cervical nucleus pulposus ablation operation.

Compared with the existing operation method of plasma scalpel head, the spinal percutaneous puncture plasma surgery operation method of the disclosure only needs to enter the cervical nucleus pulposus once, after the plasma scalpel enters the cervical nucleus pulposus, the plasma scalpel head is rotated by 360 degrees, and the rotational trajectory of the plasma scalpel head can form the first cone ablation region, the puncture needle and the plasma scalpel head are retracted by 1 mm to 2 mm, and the needle core of the plasma scalpel head is rotated by 360 degrees to form the second cone ablation region; the puncture needle and the plasma scalpel head are retracted again by 1 mm to 2 mm, and the needle core of the plasma scalpel head is rotated again by 360 degrees to form the third cone ablation region; By rotating the main ablation electrode 1 by 360 degrees at different positions to form different ablation regions, multiple entries of the plasma scalpel head in different directions may be avoided, reducing surgery time, alleviating the pain of patients and at the same time enabling to ensure the efficient work of the spinal percutaneous plasma surgery.

### Embodiment 3

The embodiment provides an operation method of a lumbar surgery performed by a plasma scalpel head for spinal percutaneous puncture, the plasma scalpel head provided in Embodiment 1 and the plasma scalpel kit are employed, and the operation method of the plasma scalpel head for spinal percutaneous puncture used in lumbar vertebrae includes the following steps.
Step S1, the puncture needle is used to be placed in the diseased lumbar intervertebral disc and then the plasma scalpel head enters the nucleus pulposus through the puncture needle.
Step S2, the plasma scalpel head and the puncture needle are locked, and the needle core of the plasma scalpel head is rotated by 360 degrees to form a first cone ablation region; and the first cone ablation region and peripheral nucleus pulposus tissue are vaporized and ablated at a low temperature.
Step S3, the puncture needle and the plasma scalpel head are retracted by 1 mm to 2 mm, and the needle core of the plasma scalpel head is rotated by 360 degrees to form a second cone ablation region; and the second cone ablation region and peripheral nucleus pulposus tissue are vaporized and ablated at a low temperature.
Step S4, the puncture needle and the plasma scalpel head are retracted again by 1 mm to 2 mm, and the needle core of the plasma scalpel head is rotated by 360 degrees to form a third cone ablation region; an ablation foot pedal is pressed to ablate the third cone ablation region and peripheral nucleus pulposus tissue.

Specifically, before the step S1, it is necessary to be in a prone posture, a Kirschner wire locates in vitro the diseased lumbar space under fluoroscopy and marks it, under the guidance of the C arm, the special puncture needle is stabbed at a distance from the midline of the affected side of 8 mm to 10 mm into the intervertebral disc through the “safety triangle” at an angle of 35 degrees to 45 degrees to the skin, the posterior lateral part of a lumbar intervertebral disc fibrous annulus is located in this area, and the surface is not covered by bony structures; in the step S1, the puncture needle should be located in the center of the nucleus pulposus and an anterior-posterior puncture needle is located in the center of the spine, and a lateral puncture needle is located in the center of the intervertebral space; the puncture needle core is pulled out and the plasma scalpel head enters the lumbar nucleus pulposus; the tip of the plasma scalpel head inserted is longer than the tip of the puncture needle, so as to ensure that the main ablation electrode 1 of the plasma scalpel head in the lumbar nucleus pulposus is not in contact with the puncture needle; the plasma scalpel head passes through the puncture needle and is gently pushed forward until a marking line reaches the tail of the puncture needle, this point is the starting point of ablation; the plasma scalpel head and a puncture cannula are fixed, the distal end of the puncture cannula is retracted into the fibrous annulus, and the plasma scalpel head is gently pushed forward until it cannot be pushed anymore, indicating that it reaches an inner edge of the fibrous annulus of the opposite side, and it is may be confirmed under fluoroscopy; at the moment, a snap ring is moved to the tail of the puncture needle, and the point is the farthest point of ablation; the scalpel head is retracted to the mark of the ablation starting point to perform ablation and formation operation.

In the step S2, after the plasma scalpel head and the puncture needle are locked, the needle core of the plasma scalpel head is rotated by 360 degrees to form the first cone ablation region; the first cone ablation region and the peripheral nucleus pulposus tissue are vaporized and ablated at a low temperature; in the step S3, the puncture needle and the plasma scalpel head are retracted by 1 mm to 2 mm, and the needle core of the plasma scalpel head is rotated by 360 degrees to form the second cone ablation region; and the second cone ablation region and the peripheral nucleus pulposus tissue are vaporized and ablated at a low temperature; in the step S4, the puncture needle and the plasma scalpel head are retracted again by 1 mm to 2 mm, and the needle core of the plasma scalpel head is rotated by 360 degrees to form the third cone ablation region; the ablation foot pedal is pressed to ablate the third cone ablation region and the peripheral nucleus pulposus tissue.

Compared with the existing operation method of plasma scalpel head, the spinal percutaneous puncture plasma surgery operation method of the disclosure only needs to enters the lumbar nucleus pulposus once, after the plasma scalpel enters the lumbar nucleus pulposus, the plasma scalpel head is rotated by 360 degrees, and the rotational trajectory of the plasma scalpel head can form the first cone ablation region, the puncture needle and the plasma scalpel head are retracted by 1 mm to 2 mm, and the needle core of the plasma scalpel head is rotated by 360 degrees to form the second cone ablation region; the puncture needle and the plasma scalpel head are retracted again by 1 mm to 2 mm, and the needle core of the plasma scalpel head is rotated again by 360 degrees to form the third cone ablation region; By rotating the main ablation electrode 1 by 360 degrees at different positions to form different ablation regions, multiple entries of the plasma scalpel head in different directions may be avoided, reducing surgery time, alleviating the pain of patients and at the same time enabling to ensure the efficient work of the spinal percutaneous plasma surgery.

The above descriptions are only preferred embodiments of the disclosure, but the scope of protection of the disclosure is not limited thereto, and any changes or substitutions that could be easily conceived of by those skilled in the art within the technical scope of the disclosure shall fall within the protection scope of the disclosure.

1-10. (canceled)

11. A plasma scalpel head for spinal percutaneous puncture, wherein the plasma scalpel head comprises a needle core; the needle core comprising a main ablation electrode and a needle core body; the main ablation electrode being arranged at an ablation end of the plasma scalpel head; the needle core body being externally provided with a first insulating layer with a same length as the needle core body; the first insulating layer being externally provided with a reflow electrode layer; a length of the reflow electrode layer being less than a length of the first insulating layer, and a part of the first insulating layer that is not covered with the reflow electrode layer forming a main ablation electrode protection insulating ring; and the reflow electrode layer being provided thereon with a second insulating layer having with a length less than the length of the reflow electrode layer, and a part of the reflow electrode layer that is not covered with the second insulating layer forming a reflow electrode.

12. The plasma scalpel head for spinal percutaneous puncture as claimed in claim 11, wherein a length of the main ablation electrode protection insulating ring is in a range of 1 mm to 1.5 mm; and an angle between the main ablation electrode and the needle core body is in a range of 10 degrees to 15 degrees.

13. The plasma scalpel head for spinal percutaneous puncture as claimed in claim 11, wherein a material of the needle core of the plasma scalpel head is stainless steel; and the ablation end of the main ablation electrode is of cylindrical-shaped.

14. The plasma scalpel head for spinal percutaneous puncture as claimed in claim 11, wherein a material of the needle core of the plasma scalpel head is tungsten steel; and the ablation end of the main ablation electrode is of cone-shaped.

15. The plasma scalpel head for spinal percutaneous puncture as claimed in claim 11, wherein a ratio of a surface area of the main ablation electrode of the plasma scalpel to a surface area of the reflow electrode of the plasma scalpel is in a range of 1:3-1:7.

16. The plasma scalpel head for spinal percutaneous puncture as claimed in claim 11, wherein both the first insulating layer and the second insulating layer are made of plastic insulating layers.

17. A plasma scalpel kit for spinal percutaneous puncture, wherein the plasma scalpel kit is used in cooperation with the plasma scalpel head as claimed in claim 11, the plasma scalpel kit comprising a puncture needle core and a puncture needle sleeved on the puncture needle core; the puncture needle being used for puncturing and being placed into a diseased site of a spine.

18. An operation method of a plasma scalpel head for spinal percutaneous puncture, using the plasma scalpel head and the plasma scalpel kit as claimed in claim 17, the operation method of the plasma scalpel head comprising following steps:
step S1, employing a puncture needle to be placed in a diseased disc and then the plasma scalpel head entering the nucleus pulposus from the puncture needle; and
step S2, locking the plasma scalpel head and the puncture needle, and rotating the needle core of the plasma scalpel head by 360 degrees to form a first cone ablation region; and vaporizing and ablating the first cone ablation region and peripheral nucleus pulposus tissue by a main ablation electrode so as to achieve intervertebral disc decompression and intervertebral disc formation.

19. The operation method of the plasma scalpel head for spinal percutaneous puncture as claimed in claim 18, further comprising: step S3, in the step S3, the puncture needle and the plasma scalpel head are retracted by 1 mm to 2 mm, and the needle core of the plasma scalpel head is rotated by 360 degrees to form a second cone ablation region; and the second cone ablation region and peripheral nucleus pulposus tissue are vaporized and ablated by the main ablation electrode so as to achieve intervertebral disc decompression and intervertebral disc formation.

20. The operation method of the plasma scalpel head for spinal percutaneous puncture as claimed in claim 19, further comprising: step S4, in the step S4, the puncture needle and the plasma scalpel head are retracted again by 1 mm to 2 mm, and the needle core of the plasma scalpel head is rotated by 360 degrees to form a third cone ablation region; and the third cone ablation region and peripheral nucleus pulposus tissue are vaporized and ablated by the main ablation electrode so as to achieve intervertebral disc decompression and intervertebral disc formation.

21. The plasma scalpel kit for spinal percutaneous puncture as claimed in claim 17, wherein a length of the main ablation electrode protection insulating ring is in a range of 1 mm to 1.5 mm; and an angle between the main ablation electrode and the needle core body is in a range of 10 degrees to 15 degrees.

22. The plasma scalpel kit for spinal percutaneous puncture as claimed in claim 17, wherein a material of the needle core of the plasma scalpel head is stainless steel; and the ablation end of the main ablation electrode is of cylindrical-shaped.

23. The plasma scalpel kit for spinal percutaneous puncture as claimed in claim 17, wherein a material of the needle core of the plasma scalpel head is tungsten steel; and the ablation end of the main ablation electrode is of cone-shaped.

24. The plasma scalpel kit for spinal percutaneous puncture as claimed in claim 17, wherein a ratio of a surface area of the main ablation electrode of the plasma scalpel to a surface area of the reflow electrode of the plasma scalpel is in a range of 1:3-1:7.

25. The plasma scalpel kit for spinal percutaneous puncture as claimed in claim 17, wherein both the first insulating layer and the second insulating layer are made of plastic insulating layers.
